(19) United States
(12) Patent Application Publication
(10) Pub. No.: US 2016/0282151 A1
(43) Pub. Date: Sep. 29, 2016

(54) INTEGRATED GAS SENSOR

(71) Applicant: Intel Corporation, Santa Clara, CA (US)

(72) Inventors: Mikko Kursula, Lempaala (FI); Marko Vaaranmaa, Lempaala (FI); Seppo Vesamaki, Tampere (FI); Timo H. Nissinen, Ylojarvi (FI); Matti A. Lahdenpera, Tampere (FI)

(21) Appl. No.: 14/666,622

(22) Filed: Mar. 24, 2015

(51) Int. Cl. G01D 11/24; G01N 19/10; G01N 33/00

(52) U.S. Cl. CPC G01D 11/245; G01N 33/0037; G01N 33/004; G01N 33/0054; G01N 33/0044; G01N 33/0047; G01N 19/10; G01N 33/0036

(57) ABSTRACT

Described is an apparatus comprising: a housing with an opening; a gas sensor positioned within the housing and displaced from an edge of the opening such that the gas sensor is not directly underneath the opening, the gas sensor operable to sense gas; and a device positioned within the housing, and operable to generate an electromechanical induced air movement such that gas is exchanged between the opening and the gas sensor. A machine-readable media is provided having machine executable instructions, that when executed cause one or more processors to perform an operation comprising: determining information associated to airflow to cause a gas sensor to sense gas, wherein the gas sensor is positioned within a housing and displaced from an edge of the opening such that the gas sensor is not directly underneath the opening; and sending the determined information to an apparatus having the gas sensor.

### BACKGROUND

Wearable computing devices are typically small computing devices operating on relatively small amounts of power. Wearable computing devices may gather information such as sensor information, perform processing functions and then convey information to a terminal computing device. The terminal computing device may be a larger device such as a notebook computer, a tablet computer, or a smart phone. The small size of wearable computing devices may result in use of these types of devices for monitoring or sensing biological and/or environmental conditions (such as gases, blood pressure, sugar level, etc.) on, in or around a person, animal, or inanimate object. A wearable computing device may communicate with a terminal computing device using low power wireless communications.

Gas sensors are used to analyze gas content in air. For gas sensors to sample gas in air, air ventilation is needed. However, when a gas sensor is integrated in a device, such as a wearable device, smart phone, tablet, etc., the device cover blocks efficient air movement, and so the functionality of the gas sensor is compromised. One way to mitigate the above problem is to place the gas sensor outside the device (e.g., on the outer surface of the wearable device). However, placing the gas sensor outside the device exposes the gas sensor to electrostatic discharge (ESD) and mechanical damage.

### BRIEF DESCRIPTION OF THE DRAWINGS

The embodiments of the disclosure will be understood more fully from the detailed description given below and from the accompanying drawings of various embodiments of the disclosure, which, however, should not be taken to limit the disclosure to the specific embodiments, but are for explanation and understanding only.

FIG. 1 illustrates an ensemble of wearable devices including a gas sensor, according to some embodiments of the disclosure.

FIG. 2 illustrates a module (or a wearable device) with one or more gas sensors and an electromechanical device for controlling airflow to the one or more gas sensors, according to some embodiments of the disclosure.

FIG. 3 illustrates a cross-section of a module (or wearable device) with a gas sensor and a speaker membrane for controlling airflow to the gas sensor, according to some embodiments of the disclosure.

FIG. 4 illustrates a flowchart of a method for remotely controlling the module having the gas sensor, according to some embodiments of the disclosure.

FIG. 5 illustrates a smart device or a computer system or a SoC (System-on-Chip) with apparatus for controlling one or more wearable devices including devices with one or more gas sensors, according to some embodiments.

### DETAILED DESCRIPTION

Some embodiments describe an apparatus which comprises a housing with an opening, and a gas sensor positioned within the housing and away from the opening. In some embodiments, the apparatus further comprises a device positioned within the housing, and operable to generate an electromechanical induced air movement (e.g., via haptic effect, speaker, fan, etc.) such that gas is exchanged between the opening and the gas sensor. In some embodiments, the device is a speaker membrane which is operable to produce audible sound. In some embodiments, the apparatus comprises logic to lower the frequency of an AC (Alternating Current) signal for the speaker membrane such that the speaker membrane vibrates without generating human audible noise. In some embodiments, the housing is part of a wearable device.

In some embodiments, the device is at least one of: a speaker membrane, a vibrating motor, a piezoelectric haptic actuator, a fan, or an air pump. In some embodiments, the apparatus comprises a processor to process data gathered by the gas sensor. In some embodiments, the apparatus comprises an antenna to transmit the processed data to another device. In one embodiment, the other device is one of: a smart phone, a tablet PC (Personal Computer), or a wireless communication enabled device such as Wireless Local Area Network (WLAN) enabled device. In some embodiments, the gas sensor is operable to sense at least one of: Nitrogen oxide gas, Carbon dioxide, Carbon monoxide, air humidity, alcohol fumes, Ozone, ammonia, formaldehyde, methane, Sulfur-dioxide, or volatile organic compound gas. In some embodiments, the apparatus comprises logic to manage airflow by the device.

In the following description, numerous details are discussed to provide a more thorough explanation of embodiments of the present disclosure. It will be apparent, however, to one skilled in the art, that embodiments of the present disclosure may be practiced without these specific details. In other instances, well-known structures and devices are shown in block diagram form, rather than in detail, in order to avoid obscuring embodiments of the present disclosure.

Note that in the corresponding drawings of the embodiments, signals are represented with lines. Some lines may be thicker, to indicate more constituent signal paths, and/or have arrows at one or more ends, to indicate primary information flow direction. Such indications are not intended to be limiting. Rather, the lines are used in connection with one or more exemplary embodiments to facilitate easier understanding of a circuit or a logical unit. Any represented signal, as dictated by design needs or preferences, may actually comprise one or more signals that may travel in either direction and may be implemented with any suitable type of signal scheme.

Throughout the specification, and in the claims, the term “connected” means a direct electrical, mechanical, or magnetic connection between the things that are connected, without any intermediary devices. The term “coupled” means either a direct electrical, mechanical, or magnetic connection between the things that are connected or an indirect connection through one or more passive or active intermediary devices. The term “circuit” or “module” may refer to one or more passive and/or active components that are arranged to cooperate with one another to provide a desired function. The term “signal” may refer to at least one current signal, voltage signal, magnetic signal, or data/clock signal. The meaning of “a,” “an,” and “the” include plural references. The meaning of “in” includes “in” and “on.”

The terms “substantially,” “close,” “approximately,” “near,” and “about,” generally refer to being within +/−20% of a target value. Unless otherwise specified the use of the ordinal adjectives “first,” “second,” and “third,” etc., to describe a common object, merely indicate that different instances of like objects are being referred to, and are not intended to imply that the objects so described must be in a given sequence, either temporally, spatially, in ranking or in any other manner.

For purposes of the embodiments, the transistors in various circuits, modules, and logic blocks are metal oxide semiconductor (MOS) transistors, which include drain, source, gate, and bulk terminals. The transistors also include Tri-Gate and FinFET transistors, Gate All Around Cylindrical Transistors, Tunneling FET (TFET), Square Wire, or Rectangular Ribbon Transistors or other devices implementing transistor functionality like carbon nano tubes or spintronic devices. MOSFET symmetrical source and drain terminals i.e., are identical terminals and are interchangeably used here. A TFET device, on the other hand, has asymmetric Source and Drain terminals. Those skilled in the art will appreciate that other transistors, for example, Bi-polar junction transistors—BJT PNP/NPN, BiCMOS, CMOS, eFET, etc., may be used without departing from the scope of the disclosure.

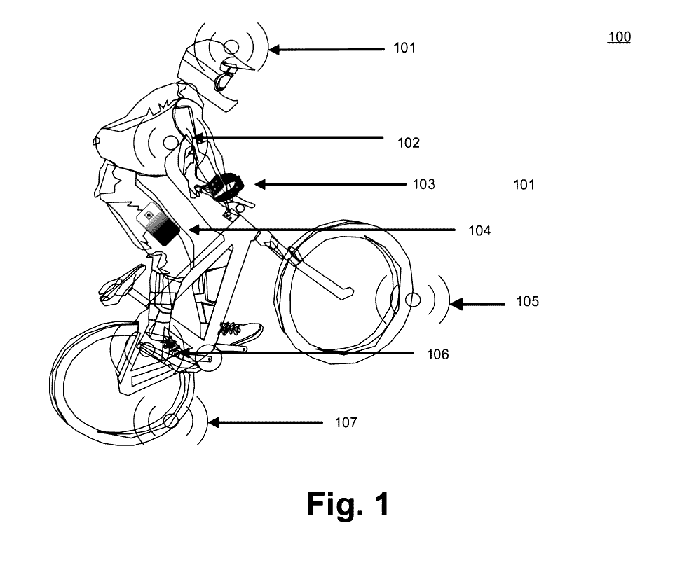

FIG. 1 illustrates ensemble 100 of wearable devices including a gas sensor, according to some embodiments of the disclosure. In this example, ensemble 100 is on a person and his ride (here, a bicycle). However, the embodiments are not limited to such. Other scenarios of wearable devices and their usage may work with various embodiments.

For example, sensors can be embedded into some other products (e.g., walls in a house, vehicles, shoes, clothes, bike tires, etc.) and can be controlled using a controller. The gas sensors of some embodiments can also be part of a wearable device. The term “wearable device” (or wearable computing device) generally refers to a device coupled to a person. For example, devices (such as sensors, cameras, microphones (mic), etc.) which are directly attached on a person or on the person's clothing are within the scope of wearable devices.

In some examples, wearable computing devices may be powered by a main power supply such as an AC/DC power outlet. In some examples, wearable computing devices may be powered by a battery. In some examples, wearable computing devices may be powered by a specialized external source based on Near Field Communication (NFC). The specialized external source may provide an electromagnetic field that may be harvested by circuitry at the wearable computing device. Another way to power the wearable computing device is electromagnetic field associated with wireless communication, for example, WLAN transmissions. WLAN transmissions use far field radio communications that have a far greater range to power a wearable computing device than NFC transmission. WLAN transmissions are commonly used for wireless communications with most types of terminal computing devices.

For example, the WLAN transmissions may be used in accordance with one or more WLAN standards based on Carrier Sense Multiple Access with Collision Detection (CSMA/CD) such as those promulgated by the Institute of Electrical Engineers (IEEE). These WLAN standards may be based on CSMA/CD wireless technologies such as WiFi™ and may include Ethernet wireless standards (including progenies and variants) associated with the IEEE 802.11-2012 Standard for Information technology—Telecommunications and information exchange between systems—Local and metropolitan area networks—Specific requirements Part 11: WLAN Media Access Controller (MAC) and Physical Layer (PHY) Specifications, published March 2012, and/or later versions of this standard (“IEEE 802.11”).

Continuing with the example of FIG. 1, ensemble 100 of wearable devices includes device 101 (e.g., camera and/or microphone) on a helmet, device 102 (e.g., blood pressure sensor, gas sensor, pulse sensor, and/or microphone, etc.) on the person's arm, device 103 (e.g., a smart watch that can function as a terminal controller or a device to be controlled), device 104 (e.g., a smart phone and/or tablet in a pocket of the person's clothing), device 105 (e.g., pressure sensor to sense or measure pressure of a tire, or gas sensor to sense nitrogen air leaking from the tire), device 106 (e.g., an accelerometer to measure paddling speed), device 107 (e.g., another pressure sensor for the other tire). In some embodiments, ensemble 100 of wearable devices has the capability to communicate by wireless energy harvesting mechanisms or other types of wireless transmission mechanisms.

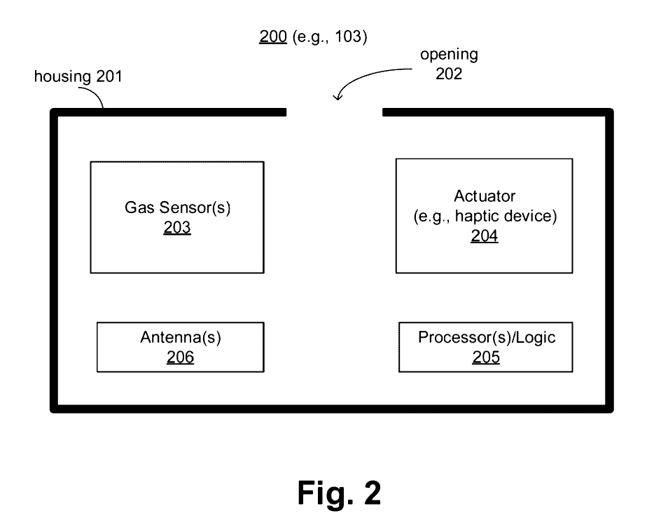

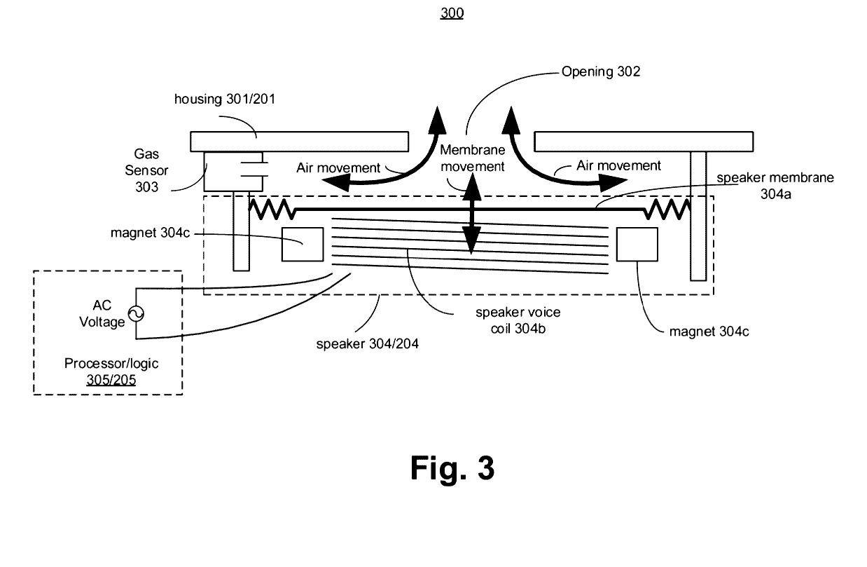

In some embodiments, device 102 comprises a housing with an opening; and a gas sensor positioned within the housing and away from the opening, where the gas sensor is operable to sense gas. In some embodiments, device 102 includes an electromechanical mechanism positioned within the housing and operable to manage airflow to the gas sensor through the opening. In some embodiments, device 102 includes a processor to process data gathered by the gas sensor. In some embodiments, device 102 includes an antenna to transmit the processed data to another device. In some embodiments, device 102 is a standalone device which senses gas (and/or other conditions) and provides results to a user. For example, device 102 may have its own processor to process the sensed gas and display it to the user via a screen display, sound beep, or by turning on/off a light. Some example embodiments of device 102 having a gas sensor are described with reference to FIGS. 2-3.

FIG. 2 illustrates a module (or a wearable device) 200 with one or more gas sensors and an electromechanical device for controlling airflow to the one or more gas sensors, according to some embodiments of the disclosure. It is pointed out that those elements of FIG. 2 having the same reference numbers (or names) as the elements of any other figure can operate or function in any manner similar to that described, but are not limited to such.

In some embodiments, module 200 comprises housing 201 with opening 202, one or more Gas Sensor(s) 203, Actuator 204 (e.g., electromechanical device), Processor(s)/Logic 205, and Antenna(s) 206. So as not to obscure the embodiments, a simplified version of module 200 is illustrated. For example, display screen, keypad, or other features may be part of module 200 but are not shown here. In some embodiments, housing 201 provides protection to the units inside housing 201. Housing 201 can be made from any suitable material. For example, housing 201 is made from plastic. In some embodiments, opening 202 is formed in housing 201 such that the one or more Gas Sensor(s) 203 are close to opening 202 but not directly under it so as to prevent harm being caused to the one or more Gas Sensor(s) 203. For example, the one or more Gas Sensor(s) 203 are positioned such that they are displaced from an edge of opening 202 so that the one or more Gas Sensor(s) 203 are not directly underneath opening 202. In some embodiments, opening 202 is covered by a mesh (e.g., a wire or plastic mesh).

In some embodiments, the one or more Gas Sensor(s) 203 are operable to sense at least one of: Nitrogen oxide gas, Carbon dioxide, Carbon monoxide, air humidity, alcohol fumes, Ozone, ammonia, formaldehyde, methane, Sulfur-dioxide, Freon, oxygen, or volatile organic compound gas. The one or more Gas Sensor(s) 203 are suitable to sense other types of gases too. In some embodiments, the apparatus comprises logic to manage airflow by the device. In some embodiments, Actuator 204 is at least one of: a speaker membrane, a vibrating motor (e.g., Vibra alert motor), a piezoelectric haptic actuator, a fan, or an air pump (e.g., piezoelectric air pumps for cooling purposes). In other embodiments, other types of actuators and/or haptic devices may be used for Actuator 204. For example, actuators which can vibrate the display glass of a wearable device (e.g., smart watch) to produce air movement can be used for Actuator 204. Other electromechanical devices may also be used as Actuator 204.

In some embodiments, Processor/Logic 205 controls Actuator 204 to manage airflow through opening 202 and around the one or more Gas Sensor(s) 203. In some embodiments, Processor/Logic 205 is a low power processor. In some embodiments, Processor/Logic 205 receives instructions (via Antenna(s) 206) from a terminal device (e.g., a smart phone, tablet, etc.) and controls the airflow for the one or more Gas Sensor(s) 203 to sample gas in air. In some embodiments, Processor/Logic 205 is preprogrammed to manage airflow within device 200. For example, in some embodiments, module 200 is a standalone device which is preprogrammed to sense particular kind(s) of gases and then report its result to a user (e.g., by a display screen, sound beep, light indicator, etc.). In some embodiments, module 200 includes a haptic feedback to report the result of the identified gas to a user.

In some embodiments, Antenna(s) 206 are provided as part of module 200 to communicate with other devices. In some embodiments, Antenna(s) 206 may comprise one or more directional or omnidirectional antennas, including monopole antennas, dipole antennas, loop antennas, patch antennas, microstrip antennas, coplanar wave antennas, or other types of antennas suitable for transmission of Radio Frequency (RF) signals. In some multiple-input multiple-output (MIMO) embodiments, Antenna(s) 206 are separated to take advantage of spatial diversity.

FIG. 3 illustrates cross-section 300 of a module (or a wearable device) with a gas sensor and a speaker membrane for controlling airflow to the gas sensor, according to some embodiments of the disclosure. It is pointed out that those elements of FIG. 3 having the same reference numbers (or names) as the elements of any other figure can operate or function in any manner similar to that described, but are not limited to such.

In some embodiments, cross-section 300 illustrates the cross-section of module 200 having housing 301/201, opening 302/202, Gas Sensor 303, Speaker as Actuator 304/204, and Processor/Logic 305/205. In some embodiments, Speaker 304 comprises speaker membrane 304a, speaker voice coil 304b and magnets 304c on either side of speaker voice coil 304b. In some embodiments, speaker membrane 304a is operable to vibrate to generate sound. Any suitable material may be used for forming speaker membrane 304a. The vibrations caused by the membrane movement cause air movement through Opening 302 and the area within housing 301 near opening 302. The air movement causes gas in air, outside housing 301, to be transported to Gas Sensor 303. As such, Gas Sensor 303 samples the gas in the air and determines the kind of specific gas.

In some embodiments, the sensed gas information is provided to Processor/Logic 304 which then sends the gas information to a terminal device (e.g., a phone) via Antenna(s) 206. In some embodiments, the sensed gas information is provided to Processor/Logic 305 which then sends the gas information to an output mechanism (e.g., display screen). In some embodiments, gas exchange takes place when Speaker 304 is normally operating. For example, when Speaker 304 is producing sound (e.g., music), module 200 (e.g., a wearable device) is also enabled to sense gas. In such embodiments, Gas Sensor 303 can directly benefit from the operation of Speaker 304 and may not need additional ventilation. In this example, the airflow is generated by speaker membrane 304a as it vibrates during its normal operation.

In some embodiments, when Speaker 304 is not operational for its primary purpose (e.g., for making sound/music), Processor/Logic 305/205 causes Speaker 304 to be driven by a very low frequency AC voltage signal (e.g., 10 Hz). In this example, Speaker 304 does not generate audible sound but produces enough vibrations to cause fresh gas exchange for Gas Sensor 302 to sample new gas molecules. While the embodiment of FIG. 3 illustrates controlling the vibration frequency of speaker membrane 304a to manage airflow, the airflow can be managed by other electromechanical devices. For example, in some embodiments, an electromechanical device is a cooling fan or a blower, and Processor/Logic 305/205 controls the speed of the cooling fan or blower to be slow enough for air exchange, for proper operation of Gas Sensor 302, but not fast enough to cause noise audible to a human.

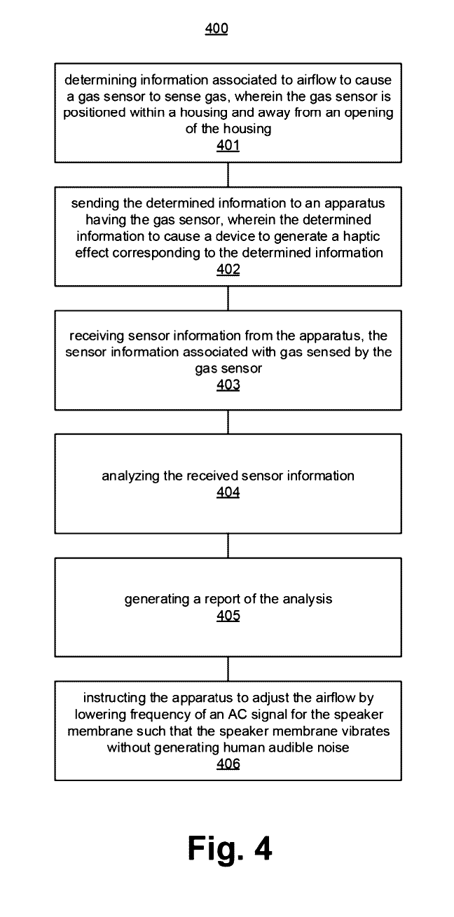

FIG. 4 illustrates flowchart 400 of a method for remotely controlling module 200, according to some embodiments of the disclosure. It is pointed out that those elements of FIG. 4 having the same reference numbers (or names) as the elements of any other figure can operate or function in any manner similar to that described, but are not limited to such.

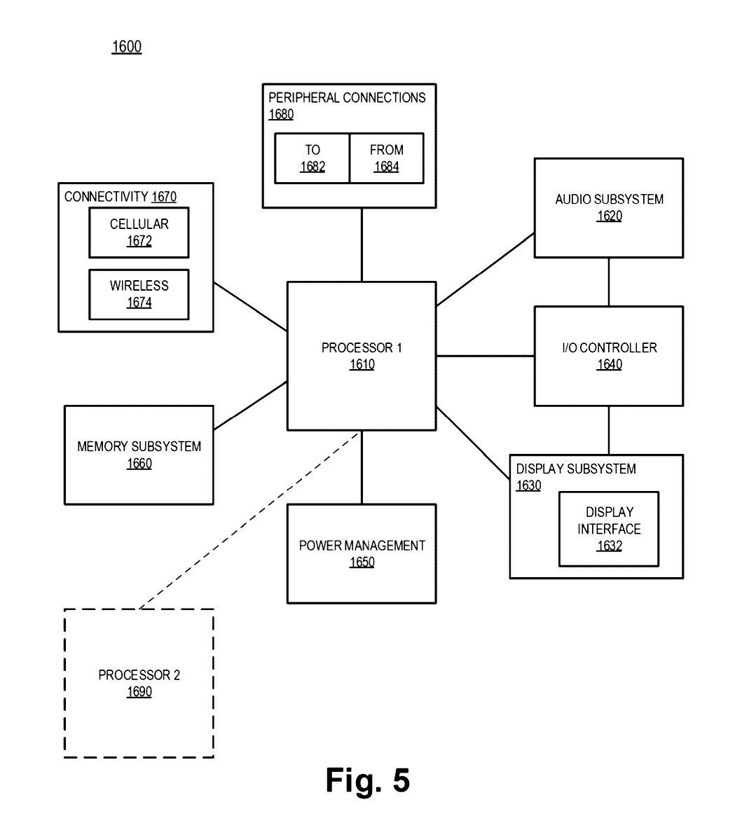

Although the blocks in the flowchart with reference to FIG. 4 are shown in a particular order, the order of the actions can be modified. Thus, the illustrated embodiments can be performed in a different order, and some actions/blocks may be performed in parallel. Some of the blocks and/or operations listed in FIG. 5 are optional in accordance with certain embodiments. The numbering of the blocks presented is for the sake of clarity and is not intended to prescribe an order of operations in which the various blocks must occur. Additionally, operations from the various flows may be utilized in a variety of combinations.

At block 401, information about airflow speed for proper function or operation of the one or more Gas Sensors 203 is determined by a terminal device (e.g., smart phone, tablet PC, etc.). For example, if one of the Gas Sensors 203, which is positioned inside housing 201 of device 200, is an alcohol sensor then higher speed air exchange is needed compared to a carbon monoxide gas sensor. If device 200 has both of these sensors, then the terminal device uses the limiting sensor's air speed requirements to set the air exchange characteristics. In this example, the terminal device uses the air exchange requirements for the alcohol sensor to set the air exchange characteristics of device 200.

At block 402, the terminal device sends the determined information to device (or module) 200 having one or more Gas Sensor(s) 203. This determined information is used by device 200 to generate an electromechanical induced air movement (e.g., via Actuator 204) corresponding to the determined information. At block 403, the terminal device receives information from the one or more Gas Sensor(s) 203 via Antenna(s) 206 of device 200. This received information is associated with the gas sensed by the one or more Gas Sensor(s) 203.

At block 404, the terminal device analyzes the received sensor information. For example, the terminal device determines the type and amount of gas sensed by the one or more Gas Sensor(s) 203, whether the one or more Gas Sensor(s) 203 are operating properly, etc. At block 405, the terminal device generates a report of the analysis. For example, the terminal device makes a graphical report of the contents of sensed gases over time, distance, and/or location. In other embodiments, other forms of reports may be generated. For example, if one of the Gas Sensor(s) 203 detects the presence of a dangerous gas, or an amount of gas above a safe threshold level for a human or an instrument, then the terminal device may send an alarm to a user (e.g., by sounding a beep, sending an email alert, sending a text alert, etc.).

At block 406, the terminal device determines that Actuator 204 is being turned off because, for example, no sound is needed to be generated by speaker 204. In some embodiments, the terminal device adjusts the airflow by lowering the frequency of an AC signal for the speaker membrane such that speaker membrane 304a vibrates without generating human audible noise.

Program software code/instructions associated with flowchart 400 and executed to implement embodiments of the disclosed subject matter may be implemented as part of an operating system or a specific application, component, program, object, module, routine, or other sequence of instructions or organization of sequences of instructions referred to as “program software code/instructions,” “operating system program software code/instructions,” “application program software code/instructions,” or simply “software” or firmware embedded in processor. In some embodiments, the program software code/instructions associated with flowchart 400 are executed by a terminal device (such as shown in FIG. 5).

Referring back to FIG. 4, in some embodiments, the program software code/instructions associated with flowchart 400 are stored in a computer executable storage medium and executed by a terminal device. Here, computer executable storage medium is a tangible machine readable medium that can be used to store program software code/instructions and data that, when executed by a computing device, causes one or more processors to perform a method(s) as may be recited in one or more accompanying claims directed to the disclosed subject matter.

The tangible machine readable medium may include storage of the executable software program code/instructions and data in various tangible locations, including for example ROM, volatile RAM, non-volatile memory and/or cache and/or other tangible memory as referenced in the present application. Portions of this program software code/instructions and/or data may be stored in any one of these storage and memory devices. Further, the program software code/instructions can be obtained from other storage, including, e.g., through centralized servers or peer to peer networks and the like, including the Internet. Different portions of the software program code/instructions and data can be obtained at different times and in different communication sessions or in the same communication session.

The software program code/instructions (associated with flowchart 400) and data can be obtained in their entirety prior to the execution of a respective software program or application by the computing device. Alternatively, portions of the software program code/instructions and data can be obtained dynamically, e.g., just in time, when needed for execution. Alternatively, some combination of these ways of obtaining the software program code/instructions and data may occur, e.g., for different applications, components, programs, objects, modules, routines or other sequences of instructions or organization of sequences of instructions, by way of example. Thus, it is not required that the data and instructions be on a tangible machine readable medium in entirety at a particular instance of time.

Examples of tangible computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., Compact Disk Read-Only Memory (CD ROMS), Digital Versatile Disks (DVDs), etc.), among others. The software program code/instructions may be temporarily stored in digital tangible communication links while implementing electrical, optical, acoustical or other forms of propagating signals, such as carrier waves, infrared signals, digital signals, etc. through such tangible communication links.

In general, a tangible machine readable medium includes any tangible mechanism that provides (i.e., stores and/or transmits in digital form, e.g., data packets) information in a form accessible by a machine (i.e., a computing device), which may be included, e.g., in a communication device, a computing device, a network device, a personal digital assistant, a manufacturing tool, a mobile communication device, whether or not able to download and run applications and subsidized applications from the communication network, such as the Internet, e.g., an iPhone®, Galaxy®, Blackberry® Droid®, or the like, or any other device including a computing device. In one embodiment, processor-based system is in a form of or included within a PDA, a cellular phone, a notebook computer, a tablet, a game console, a set top box, an embedded system, a TV, a personal desktop computer, etc. Alternatively, the traditional communication applications and subsidized application(s) may be used in some embodiments of the disclosed subject matter.

FIG. 5 illustrates a smart device or a computer system or a SoC (System-on-Chip) with apparatus for controlling one or more wearable devices including devices with one or more gas sensors, according to some embodiments. It is pointed out that those elements of FIG. 5 having the same reference numbers (or names) as the elements of any other figure can operate or function in any manner similar to that described, but are not limited to such.

FIG. 5 illustrates a block diagram of an embodiment of a mobile device in which flat surface interface connectors could be used. In some embodiments, computing device 1600 represents a mobile computing device, such as a computing tablet, a mobile phone or smart-phone, a wireless-enabled e-reader, or other wireless mobile device. It will be understood that certain components are shown generally, and not all components of such a device are shown in computing device 1600.

In some embodiments, computing device 1600 includes a first processor 1610 with apparatus for controlling one or more devices with one or more gas sensors, according to some embodiments discussed. Other blocks of the computing device 1600 may also include the apparatus for controlling one or more devices with one or more gas sensors, according to some embodiments. The various embodiments of the present disclosure may also comprise a network interface within 1670 such as a wireless interface so that a system embodiment may be incorporated into a wireless device, for example, cell phone or personal digital assistant.

In one embodiment, processor 1610 (and/or processor 1690) can include one or more physical devices, such as microprocessors, application processors, microcontrollers, programmable logic devices, or other processing means. The processing operations performed by processor 1610 include the execution of an operating platform or operating system on which applications and/or device functions are executed. The processing operations include operations related to I/O (input/output) with a human user or with other devices, operations related to power management, and/or operations related to connecting the computing device 1600 to another device. The processing operations may also include operations related to audio I/O and/or display I/O.

In one embodiment, computing device 1600 includes audio subsystem 1620, which represents hardware (e.g., audio hardware and audio circuits) and software (e.g., drivers, codecs) components associated with providing audio functions to the computing device. Audio functions can include speaker and/or headphone output, as well as microphone input. Devices for such functions can be integrated into computing device 1600, or connected to the computing device 1600. In one embodiment, a user interacts with the computing device 1600 by providing audio commands that are received and processed by processor 1610.

Display subsystem 1630 represents hardware (e.g., display devices) and software (e.g., drivers) components that provide a visual and/or tactile display for a user to interact with the computing device 1600. Display subsystem 1630 includes display interface 1632, which includes the particular screen or hardware device used to provide a display to a user. In one embodiment, display interface 1632 includes logic separate from processor 1610 to perform at least some processing related to the display. In one embodiment, display subsystem 1630 includes a touch screen (or touch pad) device that provides both output and input to a user.

I/O controller 1640 represents hardware devices and software components related to interaction with a user. I/O controller 1640 is operable to manage hardware that is part of audio subsystem 1620 and/or display subsystem 1630. Additionally, I/O controller 1640 illustrates a connection point for additional devices that connect to computing device 1600 through which a user might interact with the system. For example, devices that can be attached to the computing device 1600 might include microphone devices, speaker or stereo systems, video systems or other display devices, keyboard or keypad devices, or other I/O devices for use with specific applications such as card readers or other devices.

As mentioned above, I/O controller 1640 can interact with audio subsystem 1620 and/or display subsystem 1630. For example, input through a microphone or other audio device can provide input or commands for one or more applications or functions of the computing device 1600. Additionally, audio output can be provided instead of, or in addition to display output. In another example, if display subsystem 1630 includes a touch screen, the display device also acts as an input device, which can be at least partially managed by I/O controller 1640. There can also be additional buttons or switches on the computing device 1600 to provide I/O functions managed by I/O controller 1640.

In one embodiment, I/O controller 1640 manages devices such as accelerometers, cameras, light sensors or other environmental sensors, or other hardware that can be included in the computing device 1600. The input can be part of direct user interaction, as well as providing environmental input to the system to influence its operations (such as filtering for noise, adjusting displays for brightness detection, applying a flash for a camera, or other features).

In one embodiment, computing device 1600 includes power management 1650 that manages battery power usage, charging of the battery, and features related to power saving operation. Memory subsystem 1660 includes memory devices for storing information in computing device 1600. Memory can include nonvolatile (state does not change if power to the memory device is interrupted) and/or volatile (state is indeterminate if power to the memory device is interrupted) memory devices. Memory subsystem 1660 can store application data, user data, music, photos, documents, or other data, as well as system data (whether long-term or temporary) related to the execution of the applications and functions of the computing device 1600.

Elements of embodiments are also provided as a machine-readable medium (e.g., memory 1660) for storing the computer-executable instructions (e.g., instructions to implement any other processes discussed herein). The machine-readable medium (e.g., memory 1660) may include, but is not limited to, flash memory, optical disks, CD-ROMs, DVD ROMs, RAMs, EPROMs, EEPROMs, magnetic or optical cards, phase change memory (PCM), or other types of machine-readable media suitable for storing electronic or computer-executable instructions. For example, embodiments of the disclosure may be downloaded as a computer program (e.g., BIOS) which may be transferred from a remote computer (e.g., a server) to a requesting computer (e.g., a client) by way of data signals via a communication link (e.g., a modem or network connection).

Connectivity 1670 includes hardware devices (e.g., wireless and/or wired connectors and communication hardware) and software components (e.g., drivers, protocol stacks) to enable the computing device 1600 to communicate with external devices. The computing device 1600 could be separate devices, such as other computing devices, wireless access points or base stations, as well as peripherals such as headsets, printers, or other devices.

Connectivity 1670 can include multiple different types of connectivity. To generalize, the computing device 1600 is illustrated with cellular connectivity 1672 and wireless connectivity 1674. Cellular connectivity 1672 refers generally to cellular network connectivity provided by wireless carriers, such as provided via GSM (global system for mobile communications) or variations or derivatives, CDMA (code division multiple access) or variations or derivatives, TDM (time division multiplexing) or variations or derivatives, or other cellular service standards. Wireless connectivity (or wireless interface) 1674 refers to wireless connectivity that is not cellular, and can include personal area networks (such as Bluetooth, Near Field, etc.), local area networks (such as Wi-Fi), and/or wide area networks (such as WiMax), or other wireless communication.

Peripheral connections 1680 include hardware interfaces and connectors, as well as software components (e.g., drivers, protocol stacks) to make peripheral connections. It will be understood that the computing device 1600 could be a peripheral device (“to” 1682) to other computing devices, as well as have peripheral devices (“from” 1684) connected to it. The computing device 1600 commonly has a “docking” connector to connect to other computing devices for purposes such as managing (e.g., downloading and/or uploading, changing, synchronizing) content on computing device 1600. Additionally, a docking connector can allow computing device 1600 to connect to certain peripherals that allow the computing device 1600 to control content output, for example, to audiovisual or other systems.

In addition to a proprietary docking connector or other proprietary connection hardware, the computing device 1600 can make peripheral connections 1680 via common or standards-based connectors. Common types can include a Universal Serial Bus (USB) connector (which can include any of a number of different hardware interfaces), DisplayPort including MiniDisplayPort (MDP), High Definition Multimedia Interface (HDMI), Firewire, or other types.

Reference in the specification to “an embodiment,” “one embodiment,” “some embodiments,” or “other embodiments” means that a particular feature, structure, or characteristic described in connection with the embodiments is included in at least some embodiments, but not necessarily all embodiments. The various appearances of “an embodiment,” “one embodiment,” or “some embodiments” are not necessarily all referring to the same embodiments. If the specification states a component, feature, structure, or characteristic “may,” “might,” or “could” be included, that particular component, feature, structure, or characteristic is not required to be included. If the specification or claim refers to “a” or “an” element, that does not mean there is only one of the elements. If the specification or claims refer to “an additional” element, that does not preclude there being more than one of the additional element.

Furthermore, the particular features, structures, functions, or characteristics may be combined in any suitable manner in one or more embodiments. For example, a first embodiment may be combined with a second embodiment anywhere the particular features, structures, functions, or characteristics associated with the two embodiments are not mutually exclusive.

While the disclosure has been described in conjunction with specific embodiments thereof, many alternatives, modifications and variations of such embodiments will be apparent to those of ordinary skill in the art in light of the foregoing description. For example, other memory architectures e.g., Dynamic RAM (DRAM) may use the embodiments discussed. The embodiments of the disclosure are intended to embrace all such alternatives, modifications, and variations as to fall within the broad scope of the appended claims.

In addition, well known power/ground connections to integrated circuit (IC) chips and other components may or may not be shown within the presented figures, for simplicity of illustration and discussion, and so as not to obscure the disclosure. Further, arrangements may be shown in block diagram form in order to avoid obscuring the disclosure, and also in view of the fact that specifics with respect to implementation of such block diagram arrangements are highly dependent upon the platform within which the present disclosure is to be implemented (i.e., such specifics should be well within purview of one skilled in the art). Where specific details (e.g., circuits) are set forth in order to describe example embodiments of the disclosure, it should be apparent to one skilled in the art that the disclosure can be practiced without, or with variation of, these specific details. The description is thus to be regarded as illustrative instead of limiting.

The following examples pertain to further embodiments. Specifics in the examples may be used anywhere in one or more embodiments. All optional features of the apparatus described herein may also be implemented with respect to a method or process.

For example, an apparatus is provided which comprises: a housing with an opening; a gas sensor positioned within the housing and displaced from an edge of the opening such that the gas sensor is not directly underneath the opening, the gas sensor operable to sense gas; and a device positioned within the housing, and operable to generate an electromechanical induced air movement such that gas is exchanged between the opening and the gas sensor.

In some embodiments, the device is a speaker membrane which is operable to produce audible sound. In some embodiments, the apparatus comprises logic to lower frequency of an AC signal for the speaker membrane such that the speaker membrane vibrates without generating human audible noise. In some embodiments, the housing is part of a wearable device. In some embodiments, the device is at least one of: a speaker membrane; a vibrating motor; a piezoelectric haptic actuator; a fan; or an air pump. In some embodiments, the apparatus a processor to process data gathered by the gas sensor.

In some embodiments, the apparatus comprises an antenna to transmit the processed data to another device. In some embodiments, the other device is one of: a smart phone; a tablet PC; or a wireless communication enabled device. In some embodiments, the apparatus comprises an output mechanism to display an output of the gas sensor. In some embodiments, the output mechanism is at least one of: a display screen; a light indicator; a sound indicator; or haptic feedback. In some embodiments, the gas sensor is operable to sense at least one of: Nitrogen oxide gas; Carbon dioxide; Carbon monoxide; Air Humidity; Alcohol fumes; Ozone; Ammonia; Formaldehyde; Methane; Sulfur dioxide; or Volatile organic compound gas. In some embodiments, the apparatus comprises logic to manage airflow by the device.

In another example, a wearable device is provided which comprises: a housing with an opening; a gas sensor positioned within the housing and displaced from an edge of the opening such that the gas sensor is not directly underneath the opening, the gas sensor operable to sense gas; an electromechanical mechanism positioned within the housing and operable to manage airflow to the gas sensor through the opening; a processor to process data gathered by the gas sensor; and an antenna to transmit the processed data to another device.

In some embodiments, the electromechanical mechanism is a speaker membrane which is operable to produce audible sound. In some embodiments, the wearable device comprises logic to a lower frequency of an AC signal for the speaker membrane such that the speaker membrane vibrates without generating human audible noise. In some embodiments, the other device is one of: a smart phone; a tablet PC; or a wireless communication enabled device.

In some embodiments, the electromechanical mechanism is at least one of: a speaker membrane; a vibrating motor; a piezoelectric haptic actuator; a fan; or an air pump. In some embodiments, the gas sensor is operable to sense at least one of: Nitrogen oxide gas; Carbon dioxide; Carbon monoxide; Air Humidity; Alcohol fumes; Ozone; Ammonia; Formaldehyde; Methane; Sulfur dioxide; or Volatile organic compound gas.

In another example, a machine readable media is provided which includes machine executable instructions, that when executed cause one or more processors to perform an operation comprising: determining information associated to airflow to cause a gas sensor to sense gas, wherein the gas sensor is positioned within a housing and displaced from an edge of the opening such that the gas sensor is not directly underneath the opening; and sending the determined information to an apparatus having the gas sensor, wherein the determined information to cause a device to generate an electromechanical induced air movement corresponding to the determined information, wherein the device is part of the apparatus and positioned within the housing away from the opening.

In some embodiments, further machine executable instructions are provided that when executed cause the one or more processors to perform a further operation comprising: receiving sensor information from the apparatus, the sensor information associated with gas sensed by the gas sensor. In some embodiments, further machine executable instructions are provided that when executed cause the one or more processors to perform a further operation comprising: analyzing the received sensor information; and generating a report of the analysis.

In some embodiments, further machine executable instructions are provided that when executed cause the one or more processors to perform a further operation comprising: sending an alarm when the analysis indicates sensed gas is above a threshold. In some embodiments, the device is a speaker membrane, and wherein the machine readable media includes further machine executable instructions, that when executed cause the one or more processors to perform a further operation comprising: instructing the apparatus to adjust the airflow by lowering frequency of an AC signal for the speaker membrane such that the speaker membrane vibrates without generating human audible noise.

In another example, a method is provided which comprises: determining information associated to airflow to cause a gas sensor to sense gas, wherein the gas sensor is positioned within a housing and displaced from an edge of the opening such that the gas sensor is not directly underneath the opening; and sending the determined information to an apparatus having the gas sensor, wherein the determined information to cause a device to generate an electromechanical induced air movement corresponding to the determined information, wherein the device is part of the apparatus and positioned within the housing away from the opening.

In some embodiments, a method is provided which comprises receiving sensor information from the apparatus, the sensor information associated with gas sensed by the gas sensor. In some embodiments, the method further comprises: analyzing the received sensor information; and generating a report of the analysis. In some embodiments, a method is provided which comprises sending an alarm when the analysis indicates sensed gas is above a threshold. In some embodiments, the device is a speaker membrane. In some embodiments, the method comprises instructing the apparatus to adjust the airflow by lowering frequency of an AC signal for the speaker membrane such that the speaker membrane vibrates without generating human audible noise.

In another example, an apparatus is provided which comprises: means for determining information associated to airflow to cause a gas sensor to sense gas, wherein the gas sensor is positioned within a housing and displaced from an edge of the opening such that the gas sensor is not directly underneath the opening; and means for sending the determined information to a system having the gas sensor, wherein the determined information to cause a device to generate an electromechanical induced air movement corresponding to the determined information, wherein the device is part of the apparatus and positioned within the housing away from the opening.

In some embodiments, the apparatus further comprises: means for receiving sensor information from the system, the sensor information associated with gas sensed by the gas sensor. In some embodiments, the apparatus further comprises: means for analyzing the received sensor information; and means for generating a report of the analysis. In some embodiments, the apparatus further comprises: means for sending an alarm when the analysis indicates sensed gas is above a threshold. In some embodiments, the device is a speaker membrane. In some embodiments, the apparatus further comprises: means for instructing the system to adjust the airflow by lowering frequency of an AC signal for the speaker membrane such that the speaker membrane vibrates without generating human audible noise.

An abstract is provided that will allow the reader to ascertain the nature and gist of the technical disclosure. The abstract is submitted with the understanding that it will not be used to limit the scope or meaning of the claims. The following claims are hereby incorporated into the detailed description, with each claim standing on its own as a separate embodiment.

We claim:

1. An apparatus comprising:
a housing with an opening;
a gas sensor positioned within the housing and displaced from an edge of the opening such that the gas sensor is not directly underneath the opening, the gas sensor operable to sense gas; and
a device positioned within the housing, and operable to generate an electromechanical induced air movement such that gas is exchanged between the opening and the gas sensor.

2. The apparatus of claim 1, wherein the device is a speaker membrane which is operable to produce audible sound.

3. The apparatus of claim 2 comprises logic to lower frequency of an AC signal for the speaker membrane such that the speaker membrane vibrates without generating human audible noise.

4. The apparatus of claim 1, wherein the housing is part of a wearable device.

5. The apparatus of claim 1, wherein the device is at least one of:
a speaker membrane;
a vibrating motor;
a piezoelectric haptic actuator;
a fan; or
an air pump.

6. The apparatus of claim 1 comprises a processor to process data gathered by the gas sensor.

7. The apparatus of claim 6 comprises an antenna to transmit the processed data to another device.

8. The apparatus of claim 6, wherein the other device is one of:
a smart phone;
a tablet PC; or
a wireless communication enabled device.

9. The apparatus of claim 1 comprises an output mechanism to display an output of the gas sensor.

10. The apparatus of claim 9, wherein the output mechanism is at least one of:
a display screen;
a light indicator;
a sound indicator; or
haptic feedback.

11. The apparatus of claim 1, wherein the gas sensor is operable to sense at least one of:
Nitrogen oxide gas;
Carbon dioxide;
Carbon monoxide;
Air Humidity;
Alcohol fumes;
Ozone;
Ammonia;
Formaldehyde;
Methane;
Sulfur dioxide; or
Volatile organic compound gas.

12. The apparatus of claim 1 comprises logic to manage airflow by the device.

13. A wearable device comprising:
a housing with an opening;
a gas sensor positioned within the housing and displaced from an edge of the opening such that the gas sensor is not directly underneath the opening, the gas sensor operable to sense gas;
an electromechanical mechanism positioned within the housing and operable to manage airflow to the gas sensor through the opening;
a processor to process data gathered by the gas sensor; and
an antenna to transmit the processed data to another device.

14. The wearable device of claim 13, wherein the electromechanical mechanism is a speaker membrane which is operable to produce audible sound.

15. The wearable device of claim 14 comprises logic to a lower frequency of an AC signal for the speaker membrane such that the speaker membrane vibrates without generating human audible noise.

16. The wearable device of claim 13, wherein the other device is one of:
a smart phone;
a tablet PC; or
a wireless communication enabled device.

17. The wearable device of claim 13, wherein the electromechanical mechanism is at least one of:
a speaker membrane;
a vibrating motor;
a piezoelectric haptic actuator;
a fan; or
an air pump.

18. The wearable device of claim 13, wherein the gas sensor is operable to sense at least one of:
Nitrogen oxide gas;
Carbon dioxide;
Carbon monoxide;
Air Humidity;
Alcohol fumes;
Ozone;
Ammonia;
Formaldehyde;
Methane;
Sulfur dioxide; or
Volatile organic compound gas.

19. A machine readable media having machine executable instructions, that when executed cause one or more processors to perform an operation comprising:
determining information associated to airflow to cause a gas sensor to sense gas, wherein the gas sensor is positioned within a housing and displaced from an edge of the opening such that the gas sensor is not directly underneath the opening; and
sending the determined information to an apparatus having the gas sensor, wherein the determined information to cause a device to generate an electromechanical induced air movement corresponding to the determined information, wherein the device is part of the apparatus and positioned within the housing away from the opening.

20. The machine readable media of claim 19 having further machine executable instructions, that when executed cause the one or more processors to perform a further operation comprising:
receiving sensor information from the apparatus, the sensor information associated with gas sensed by the gas sensor.

21. The machine readable media of claim 20 having further machine executable instructions, that when executed cause the one or more processors to perform a further operation comprising:
analyzing the received sensor information; and
generating a report of the analysis.

22. The machine readable media of claim 21 having further machine executable instructions, that when executed cause the one or more processors to perform a further operation comprising:
sending an alarm when the analysis indicates sensed gas is above a threshold.

23. The machine readable media of claim 19, wherein the device is a speaker membrane, and wherein the machine readable media includes further machine executable instructions, that when executed cause the one or more processors to perform a further operation comprising:
instructing the apparatus to adjust the airflow by lowering frequency of an AC signal for the speaker membrane such that the speaker membrane vibrates without generating human audible noise.
